(19) United States
(12) Patent Application Publication
(10) Pub. No.: US 2012/0220630 A1
(43) Pub. Date: Aug. 30, 2012

(54) PRUCALOPRIDE ORAL SOLUTION

(75) Inventor: Roger Carolus Augusta Embrechts, Turnhout (BE)

(73) Assignee: Shire-Movetis NV

(21) Appl. No.: 13/407,104

(22) Filed: Feb. 28, 2012

(30) Foreign Application Priority Data
Feb. 28, 2011 (GB) GB 1103397.4

(51) Int. Cl. A61K 31/4525; A61P 13/02

(52) U.S. Cl. 514/320

(57) ABSTRACT

The present invention is concerned with an oral aqueous solution comprising prucalopride or pharmaceutically acceptable acid addition salts thereof having good organoleptic properties.

### FIELD OF THE INVENTION

The present invention concerns an oral aqueous solution comprising prucalopride or pharmaceutically acceptable acid addition salts thereof having good organoleptic properties and an enhanced stability at a pH of between and about 5.0 to 7.0.

### BACKGROUND TO THE INVENTION

Prucalopride, which is the generic name for the (1:1) succinic acid addition salt of 4-amino-5-chloro-2,3-di-hydro-N-[r-(3-methoxypropyl)-4-piperidinyl]-7-benzo-furan-carboxamide, has enterokinetic properties, i.e. it has strong gastrointestinal prokinetic activity.

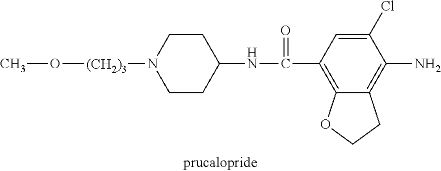

Prucalopride facilitates both cholinergic and non-cholinergic non-adrenergic (NANC) excitatory neurotransmission and stimulates colonic motility and defecation in animals. It has no affinity for 5-HT2A and 5-HT3 receptors but is a potent and selective agonist of 5-HT4 receptors. Prucalopride induces giant contractions in the colon that are propagated over the length of the colon as a peristaltic wave and therefore has significant motility enhancing effects on the large intestine.

Formulations comprising prucalopride are believed of potential use in the treatment of conditions associated with a poorly functioning bladder such as, e.g. urinary incontinence or urinary retention.

Prucalopride is generically described in EP-0,445,862-A1, published on 11 Sep. 1991, and is specifically disclosed in WO-96/16060, published on 30 May 1996.

Administration of an oral dosage form is the preferred route of administration for many pharmaceuticals because it provides for easy, low-cost administration. However some patients such as children or elderly people can have problems when requested to swallow a solid formulation such as a tablet or a capsule. Hence the development of a liquid oral formulation is therefore desirable since it offers improved patient compliance.

EP-0,445,862-A1 discloses an oral solution for use with the compounds provided in said specification. However, when an aqueous oral solution comprising prucalopride was prepared in accordance with example 22, p. 36, of EP-0,445,862-A2, i.e. comprising methylbenzoate and propylbenzoate as preservatives at a pH of 4; sorbitol as bulk sweetener; sodium saccharine as intense sweetener; and about 1 mg/ml of AI, an oral solution with undesired organoleptic properties was obtained (see page 6 line 19-page 7 line 10 of PCT Publication WO 00/66170). In PCT Publication WO 00/66170 such an oral solution was administered to a test group of 24 human volunteers in a blind study, and found to have undesirable organoleptic properties, in particular most volunteers experienced an anaesthetizing feeling on the tongue.

In order to address the aforementioned organoleptic problem and in particular the anaesthetizing feeling, an alternative oral solution comprising benzoic acid as a preservative instead of the aforementioned parabens, has been presented in said PCT publication. In as far this alternative oral solution comprising benzoic acid addresses the organoleptic problem with the former oral solution as originally disclosed in EP-0,445,862-A1 recent recommendations of health authorities required further amendments in oral solutions of prucalopride, in particular when used for pediatric applications.

In view of an immature metabolism in infancy, young pediatric patients are unable to conjugate benzoic acid efficiently, leading to an accumulation of the product that is toxic and may cause for example jaundice in said pediatric patients. It is accordingly desirable to avoid benzoic acid as preservative in medicinal products (EMEA Reflection Paper: Formulations of choice for the pediatric population—Page 23-4.1—EMEA/CHMP/PEG/194810/2005).

Also the use of mono-saccharide sweeteners like sorbitol and xylitol are faced with problems when used in pediatric formulations. Said mono-saccharides may cause osmotic diarrhea and when used orally at high doses may cause laxative effects. It is accordingly desirable to avoid mono-saccharides like sorbitol and xylitol from medicinal formulation, especially when the intended use includes pediatric patients. (EMEA Reflection Paper: Formulations of choice for the pediatric population—Page 23-4.2—EMEA/CHMP/PEG/194810/2005). In addition, when used in combination with a low pH, the high concentration in saccharides may result in an undesired risk factor for dental problems. As evident from the PCT publication WO00/66170—see page 2 lines 30-32—the currently available oral solutions for prucalopride all have a pH in the lower range of about 4, and apply mono-saccharide sweeteners. It is accordingly desirable to avoid high concentrations of monosaccharides and low pH in liquid medicinal formulations, especially when the intended use includes pediatric patients.

Even the use of intense sweeteners instead, such as sodium saccharine and aspartame, does not always result in a successful masking of the bitter taste of the active ingredient. When used in high concentrations, said intense sweeteners are known to develop a bitter aftertaste, causing undesired organoleptic properties, in particular when used in pediatric formulations.

In an effort to address the aforementioned problems associated with the known prucalopride oral solutions, the present invention provides a new oral aqueous solution having a pH ranging from 5 to 7 comprising as active ingredient prucalopride, or a pharmaceutically acceptable acid addition salt thereof; and one or more parabens. In a particular embodiment further characterized in that no intense sweeteners and/or flavors are desired.

### DETAILED DESCRIPTION OF THE INVENTION

The term prucalopride as used herein comprises the free base form and the pharmaceutically acceptable acid addition salts thereof. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic, malonic, succinic, maleic, fumaric, malic, tartaric, citric, methanesulfonic, ethanesulfonic, benzenesulfonic, p-toluenesulfonic, cyclamic, salicylic, p-aminosalicylic, pamoic and the like acids. The term addition salt as used hereinabove also comprises the solvates which prucalopride as well as the salts thereof, are able to form. Such solvates are for example hydrates, alcoholates and the like.

Preferred pharmaceutically acceptable acid addition salts of 4-amino-5-chloro-2,3-dihydro-N-[1-(3-methoxypropyl)-4-piperidinyl]-7-benzofuran-carboxamide are the hydrochloric acid (1:1) addition salt and the succinic acid (1:1) addition salt.

The solutions according to the present invention have a pH from 5 to 7, preferably from 5.5 to 6.5, most preferably about 6. The pH of the compositions is maintained by a buffer system. Buffer systems comprise mixtures of appropriate amounts of an acid such as phosphoric, succinic, tartaric, lactic, or citric acid, and a base, in particular sodium hydroxide or disodium hydrogen phosphate. Ideally, the buffer has sufficient capacity to remain in the intended pH range upon dilution with a neutral, a slightly acidic or a slightly basic beverage.

Preservatives are included in preparations to kill or inhibit the growth of microorganisms inadvertently introduced during manufacture or use and are therefore essential ingredients. The choice of a suitable preservative for a preparation depends on pH, compatibility with other ingredients, the route of administration, dose and frequency of administration of the preparation, partition coefficients with ingredients and containers or closures, degree and type of contamination, concentration required, and rate of antimicrobial effect.

In an effort to replace benzoic acid as a preservative in the earlier formulation, it has now been found, and as apparent from example 3 below, that compared to the use of benzoic acid as a preservative, the use of parabens at a pH from about and between 5 to 7, in particular from about and between 5.5 to 6.5 enhances the long-term shelf life stability of prucalopride. Prucalopride degradation products found at an ambient temperature (25° C.) after 3 months in the benzoic acid formulation are lacking in the paraben based formulation. In addition, when presented to a taste panel of 20 test persons, none of them reported the previously observed sensitizing effect of parabens on the tongue at a pH of about 4.0 (see page 7 lines 8-10 of the PCT publication WO 00/66170).

In a further aspect of the present invention, the oral aqueous solution further comprises pharmaceutically acceptable sweeteners preferably at least one intense sweetener such as saccharin, sodium or calcium saccharin, aspartame, acesulfame potassium, sodium cyclamate, alitame, a dihydrochalcone sweetener, monellin, stevioside or sucralose (4,1′, 6′-trichloro-4,1′, 6′-trideoxygalactosucrose), preferably saccharin, sodium or calcium saccharin, and optionally a bulk sweetener such as sorbitol, mannitol, fructose, sucrose, maltose, isomalt, glucose, hydrogenated glucose syrup, xylitol, caramel or honey. In particular, said sweetener is not a saccharin, like sodium or calcium saccharin, nor a bulk sweetener selected from sorbitol or xylitol. In one particular embodiment said sweetener is sucralose.

Again, compared to the prior art prucalopride formulations (supra), and as evident from example 1 below, with the use of sucralose as intense sweetener no further sweeteners/flavors are required to obtain an organoleptically acceptable formulation. As such the bitter aftertaste of intense sweeteners like sodium saccharine (see also table 2 below) can be avoided.

If employed, the intense sweetener is present in low concentrations. For example, in the case of sodium saccharin, the concentration may range from 0.01% to 0.1% (w/v) based on the total volume of the final formulation, and preferably is about 0.05% (w/v).

The bulk sweetener, and in particular sucralose, can effectively be used in larger quantities ranging from about 0.5 to 50 mg/ml, preferably from about 0.5 to 10 mg/ml, more preferably from about 1 to 5 mg/ml, even more preferably about 1 mg/ml.

In an even further aspect of the present invention, the oral aqueous solution may further comprise pharmaceutically acceptable flavours. Said masking flavours are preferably fruit flavours such as cherry, raspberry, black currant, strawberry flavour, caramel chocolate flavour, mint cool flavour, fantasy flavour and the like pharmaceutically acceptable strong flavours.

Each flavour may be present in the final composition in a concentration ranging from about 0.5 to 50 mg/ml, preferably from about 0.5 to 10 mg/ml, more preferably from about 1 to 5 mg/ml, even more preferably about 2 mg/ml.

The subject solutions may be presented in art-known containers such as bottles, spray devices, sachets, and the like. Optionally, the solutions are manufactured in unit-dose containers, e.g. unit-dose sachets or unit-dose bottles.

Further, the present invention relates to the preparation of the described solutions. The preparation involves the intimate mixing of the active ingredient with the carrier M ingredients.

In general it is contemplated that a therapeutically effective amount of prucalopride would be from about 0.001 mg/kg to about 1 mg/kg body weight, preferably from about 0.01 mg/kg to about 0.5 mg/kg body weight. A method of treatment may also include administering prucalopride on a regimen of between two or four intakes per day.

The amount of prucalopride, or a pharmaceutically acceptable acid addition salt thereof, required as daily dose in treatment will vary not only with the route of administration, the nature of the condition being treated and the age, weight and condition of the patient and will ultimately be at the discretion of the attendant physician. In general, however, a suitable daily dose will be in the range of from about 0.05 to about 50 mg per day, in particular from about 0.1 to 20 mg per day, more particular from about 0.5 to 10 mg per day, preferably from 1 to 2 mg per day. A suitable daily dose for use in prophylaxis will generally be in the same range. It may be appropriate to administer the required dose as two, three, four or more sub-doses at appropriate intervals throughout the day.

Administration can be before or after the intake of food (i.e. preprandial or postprandial).

In another aspect the present invention further provides the use of the formulation according to the present invention in the manufacturing of capsules, such as for example liquid filled and sealed hard gelatin capsules, containing said formulation. Accordingly, in one aspect the present provides capsules, comprising the aqueous formulation as described herein.

This invention will be better understood by reference to the Experimental Details that follow, but those skilled in the art will readily appreciate that these are only illustrative of the invention as described more fully in the claims that follow thereafter. Additionally, throughout this application, various publications are cited. The disclosure of these publications is hereby incorporated by reference into this application to describe more fully the state of the art to which this invention pertains.

### EXAMPLES

The objective of this study was to develop an oral solution containing Prucalopride (R108512) having a high stability of the active ingredient and being suitable for administration to infants (minimum age of 1 month) as well as adults.

About 10 years ago, an oral solution containing eq. 0.2 mg/ml Prucalopride was developed (see table 1).

TABLE 1
Formulation of R108512-F002

| | Purpose | Amount | |
|---|---|---|---|
| R108512 | Active ingredient | 0.264 | mg |
| Sorbitol 70% | Sweetening agent | 230 | μl |
| Benzoic acid | Antimicrobial preservative | 1.5 | mg |
| Strawberry | Flavoring agent | 3 | mg |
| flavor | | | |
| Sodium | Sweetening agent | 0.5 | mg |
| saccharinate | | | |
| Sodium | Alkalizing agent | q.s. ad pH 4.0 | |
| hydroxide | | | |
| Purified | Vehicle | q.s. ad 1 | ml |
| Water | | | |

However, in view of the applicability of such a formulation in small children, the use of benzoic acid can be questioned. Furthermore, in order to enable accurate dosing also in patients having a low body weight (e.g. 5 kg), an increased concentration of the active ingredient is desirable.

Taking these issues into account the use of parabens instead of benzoic acid in combination with a panel of sweeteners and/or flavors was evaluated in view of
the taste of the solution,
the antimicrobial effectiveness, and
the stability of the active ingredient:

### 1. Evaluation of Taste

In a first objective, different solutions containing a certain amount of parabens in combination with one or more sweeteners were evaluated for their taste. The results of this test are shown in table 2.

TABLE 2
Results of taste test

| Methylparaben/ | Sweetener/ | |
|---|---|---|
| propylpraben | flavor | Evaluation |
| 1.8 mg/ml | 1 mg/ml | Slightly sweet, not bitter |
| 0.2 mg/ml | sucralose | |
| 1.8 mg/ml | 2 mg/ml | Slightly sweet, not bitter |
| 0.2 mg/ml | sucralose | |
| 1.8 mg/ml | 4 mg/ml | Sweeter than 2 mg/ml |
| 0.2 mg/ml | sucralose | sucralose, not too sweet |
| 1.8 mg/ml | 0.5 mg/ml | Slightly sweet, slightly |
| 0.2 mg/ml | Na Sacch | bitter |
| 1.8 mg/ml | 0.5 mg/ml | Sweet, not bitter, slight |
| 0.2 mg/ml | Na Sacch + | aftertaste |
| | 230 μl | |
| | sorbitol | |
| | 70% | |
| 1.8 mg/ml | 1 mg/ml | Slightly sweet, light flavor |
| 0.2 mg/ml | sucralose + | taste |
| | 2 mg/ml | |
| | strawberry | |
| | cream | |
| 1.8 mg/ml | 2 mg/ml | Not sweet, light flavor |
| 0.2 mg/ml | strawberry | taste, not bitter |
| | cream | |
| 1.8 mg/ml | 1 mg/ml | Sweet, light flavor taste |
| 0.2 mg/ml | sucralose + | |
| | 2 mg/ml | |
| | PHL-131985 | |
| 1.8 mg/ml | 2 mg/ml | Not sweet, very light flavor |
| 0.2 mg/ml | PHL-131985 | taste, slightly bitter |

Further organoleptic properties were tested in a taste panel composed of 20 adults. The test persons were not allowed to have taken drinks, food or cigarettes within 15 minutes before tasting. They were asked to place two drops of the following test formulations (0.1 ml) on their tongue and to score the taste of the formulation on a scale of 0 to 100 for sweetness, bitterness, flavor and overall acceptance.

| | | | | | | Masking | |
|---|---|---|---|---|---|---|---|
| | Benzoic | Methylparaben/ | | Sodium | Sorbitol | flavor | Strawberry |
| Addition | acid | propylparaben | Sucralose | saccharinate | 70% | PHL-131985 | cream |
| B | 1.5 | — | — | 0.5 | — | — | — |
| D | 1.5 | — | 1 | — | — | — | — |
| G | 1.5 | — | — | 0.5 | 230 | — | — |
| I | 1.5 | — | 4 | — | — | — | — |
| K | — | 1.8/0.2 | 1 | — | — | — | — |
| E | — | 1.8/0.2 | 4 | — | — | — | — |
| H | — | 1.8/0.2 | — | — | — | — | 3 |
| M | — | 1.8/0.2 | — | — | — | 3 | — |
| F | — | 1.8/0.2 | 1 | — | — | — | 3 |
| J | — | 1.8/0.2 | 1 | — | — | 3 | — |

Different samples were evaluated at least 2 hours apart and not more than 2 or 3 samples were evaluated per day (see Table 3 below).

TABLE 3
Test results Taste Panel: numbers on scale from 0 to 100 and (standard deviation)

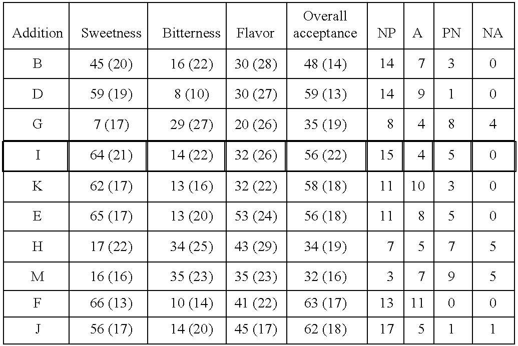

### Conclusions:

As evident from tables 2 and 3, none of the prepared formulations is considered to be unacceptable regarding their taste. Furthermore, also the formulation in which no additional flavors (additions K, E) have been added is still acceptable, and better accepted when compared to the benzoic acid formulation only comprising a sweetening agent (addition G). The sole addition of flavors to the paraben formulations (additions H, M) results in a formulation with a moderate and undesirable bitterness for the application in small children. Given the overall acceptance of the paraben formulations lacking additional flavors (additions K, E), for the application in small children it may be desirable to add one or more flavors, to make the solution sweeter.

### 2. Evaluation of Antimicrobial Effectiveness

An antimicrobial effectiveness test was performed comparing the solutions containing 1.5 mg/ml benzoic acid (at pH 3.5, 4.0 and 4.5; i.e within the acceptable pH range) with those containing 1.8 mg/ml methylparaben and 0.2 mg/ml propylparaben (at pH 5.5, 6.0 and 6.5; i.e within the acceptable pH range). The log reduction of microbial growth after 14 and 28 days are shown in tables 4 and 5.

TABLE 4
antimicrobial effectiveness test for formulations
containing 1.5 mg/ml benzoic acid

| | | pH 3.5 | | pH 4.0 | | pH 4.5 | |
|---|---|---|---|---|---|---|---|
| | | 14 | 28 | 14 | 28 | 14 | 28 |
| Organism | | days | days | days | days | days | days |
| E. coli | 9.3E5 | x | x | x | x | x | x |
| S. aureus | 2.18E6 | x | x | x | x | x | x |
| P. aeruginosa | 9.4E5 | x | x | x | x | x | x |
| S. aureus (EI*) | 6.75E5 | x | x | x | x | x | x |
| C. albicans | 1.23E6 | x | x | x | x | x | x |
| A. niger | 4.85E5 | x | x | 3.7 | x | 1.3 | 3.3 |

x = below the detection limit

TABLE 5
antimicrobial effectiveness test for formulations containing
1.8 mg/ml methylparaben and 0.2 mg/ml propylparaben

| | | pH 5.5 | | pH 6.0 | | pH 6.5 | |
|---|---|---|---|---|---|---|---|
| | | 14 | 28 | 14 | 28 | 14 | 28 |
| Organism | Blank | days | days | days | days | days | days |
| E. coli | 1.26E6 | x | x | x | x | x | x |
| S. aureus | 2.16E6 | x | x | x | x | x | x |
| P. aeruginosa | 1.11E6 | x | x | x | x | x | x |
| S. aureus (EI*) | 1.38E6 | x | x | x | x | x | x |
| C. albicans | 1.2E6 | x | x | x | x | x | x |
| A. niger | 4.05E5 | x | x | 3.9 | x | 2.7 | x |

x = below the detection limit

As evident from tables 4 and 5, all formulations have a good antimicrobial activity for most of the tested microbial strains, within their corresponding pH ranges. Furthermore, all formulations meet the requirements of the antimicrobial effectiveness test of the European Japanese and US pharmacopeia.

### 3. Evaluation of Stability of the Active Ingredient

Stability studies were performed with 1.5 mg/ml benzoic acid at pH 4.0 and 1.8 mg/ml methylparaben+0.2 mg/ml propylparaben at pH 6.0.

TABLE 6
formulations used for stability testing:

| Excipient, | | | | |
|---|---|---|---|---|
| Concentration, | | | | |
| mg/ml | Benzoic acid | | Parabens | |
| R108512 | 0.66 | mg/ml | 0.66 | mg/ml |
| Sodium benzoate | 1.77 | mg/ml | | |
| Sucralose | 1 | | 1 | |
| Methylparaben | | | 1.8 | mg/ml |
| Propylparaben | | | 0.2 | mg/ml |
| Hydrochloric acid | q.s. ad pH 4.0 | | | |
| Sodium hydroxide | | | q.s. ad pH 6.0 | |
| Purified water | q.s. ad 1 | ml | q.s. ad 1 | ml |

### Manufacturing:

1: dissolve parabens (by heating to 95-100° C.) or sodium benzoate in approximately 90% of the water by stirring
2: dissolve sucralose at room temperature while stirring in (1)
3: dissolve prucalopride at room temperature while stirring in (2)
4: adjust the pH to 3.9-4.1 by adding hydrochloric acid for the benzoic acid solution and to 5.9-6.1 by adding sodium hydroxide
5: add water to obtain the final volume.
6: fill in 30 ml amber glass bottles with polyprop screw cap.

### Results:

Results after 3 months stability at 25, 40 and 50° C. are reported below.

| | | | Total | Comp. (HPLC) (%) | | | |
|---|---|---|---|---|---|---|---|
| Long term stability of | | | Degradants | Benzoic Acid | Propylparaben | Methylparaben | Prucalopride |
| Prucalopride oral solutions | | Timepoint | % (w_w) | % (w_w) | % (w_w) | % (w_w) | % by weigh |
| Benzoic Acid (pH 4.0) | | Initial | <0.10 | 101.6 | | | 101.8 |
| | 25° C./60% RH | 1 month | <0.10 | 98.5 | | | 99.7 |
| | | 3 months | 0.14 | 98.2 | | | 98.2 |
| | 40° C./75% RH | 1 month | <0.10 | 98.9 | | | 99.6 |
| | | 3 months | <0.10 | 98.3 | | | 98.8 |
| | 50° C. | 1 month | <0.10 | 97.8 | | | 98.5 |
| | | 3 months | <0.10 | 98.5 | | | 99.4 |
| Parabens (pH 6.0) | | Initial | 0.16 | | 97.8 | 98.1 | 98.1 |
| | 25° C./60% RH | 1 month | 0.29 | | 97.8 | 100.1 | 100.5 |
| | | 3 months | 1.3 | | 96.8 | 96.2 | 99.5 |
| | 40° C./75% RH | 1 month | 0.82 | | 96.4 | 99.0 | 99.8 |
| | | 3 months | 6.3 | | 96.9 | 98.9 | 100.2 |
| | 50° C. | 1 month | 2.1 | | 96.0 | 98.8 | 100.6 |
| | | 3 months | 18.2 | | 96.3 | 99.0 | 100.3 |

### Conclusions:

BENZOIC ACID
Assay prucalopride and benzoic acid are at target
Degradants of prucalopride are present at 25° C. and 40° C. at low concentrations and are not present at 50° C.

PARABENS
Assay prucalopride is at target
Methylparaben is at target
There is a slight decrease of propylparaben (96%) at 50° C. but it remain well within specification
At 50° C. a paraben degradant is observed. When calculated against propylparaben the concentration of the degradant is 2-3% which is in line with the slight decrease of the assay of propylparaben
No degradants of prucalopride are present at any of the tested temperatures.

### 4. Final Compositions and Manufacturing Method of the New Prucalopride Oral Solution

TABLE 6
Composition of the formulations (conc in mg/ml)

| | | Active | Active | Active | Placebo |
|---|---|---|---|---|---|
| Code | Excipients | solution | solution | solution | solution |
| 073255 | R108512 | 0.66 | 0.528 | 0.396 | — |
| | (prucalopride) | | | | |
| 503581 | Sucralose | 1 | 1 | 1 | 0.5 |
| 006275 | Methylparaben | 1.8 | 1.8 | 1.8 | 1.8 |
| 007737 | Propylparaben | 0.2 | 0.2 | 0.2 | 0.2 |
| 655006 | Hydrochloric | q.s. ad | q.s. ad | q.s. ad | q.s. ad |
| | acid | pH 6.0 | pH 6.0 | pH 6.0 | pH 6.0 |
| 006789 | Sodium | q.s. ad | q.s. ad | q.s. ad | q.s. ad |
| | hydroxide | pH 6.0 | pH 6.0 | pH 6.0 | pH 6.0 |
| — | Purified water | q.s. ad | q.s. ad | q.s. ad | q.s. ad |
| | | 1 ml | 1 ml | 1 ml | 1 ml |

### Manufacturing:

1: dissolve the parabens in water by stirring while heating
2: cool down solution (1)
3: dissolve sucralose (in 2) at room temperature while stirring
4: dissolve prucalopride in (3) at room temperature while stirring
5: adjust the pH to 5.9-6.1 by adding hydrochloric acid or sodium hydroxide
6: add water to obtain the final volume
7: fill in bottles

Remark: it is not crucial that sucralose or prucalopride is added first or second

### Characteristics:

pH: target: 6.0±0.1, limits: 5.5-6.5
Density: 1.001 at 21.6° C.

1. An oral aqueous solution having a pH ranging from about 5 to about 7 comprising as an active ingredient prucalopride, or a pharmaceutically acceptable acid addition salt thereof; and one or more parabens.

2. The oral solution according to claim 1, wherein the amount of the one or more parabens ranges from about 0.1 mg/ml to about 2 mg/ml for each paraben.

3. The oral solution according to claim 1, wherein the one or more parabens are selected from the group consisting of methylparaben, ethylparaben, propylparaben, butylparaben, isobutylparaben, isopropylparaben, and benzylparaben.

4. The oral solution according to claim 1, wherein the pharmaceutically acceptable addition salt of prucalopride is the (1:1) succinic acid addition salt.

5. The oral solution according to claim 1, wherein the pharmaceutically acceptable addition salt of prucalopride is the (1:1) hydrochlorid acid addition salt.

6. The oral solution according to claim 1, comprising methylparaben and propylparaben.

7. The oral solution according to claim 6, comprising between and about 1.5-about 2.5 mg/ml methylparaben; and between and about 0.15-about 0.25 mg/ml propylparaben.

8. The oral solution according to claim 1, wherein the pH ranges of from about 5.5 to about 6.5; in particular has a pH of about 6.0.

9. The oral solution according to claim 1, further comprising between and about 0.5 to about 10 mg/ml sucralose, in particular about 1 mg/ml.

10. The oral solution according to claim 1 comprising the following ingredients:
between and about 0.3-about 0.7 mg/ml prucalopride succinic acid (1:1) addition salt;
about 1 mg/ml sucralose;
between and about 1.5-about 2.5 mg/ml methylparaben;
between and about 0.15-about 0.25 mg/ml propylparaben; and
Hydrochloric acid and/or Sodium hydroxide to adjust the pH to about 6.

11. A method of preparing an oral aqueous solution and comprising as an active ingredient prucalopride or a pharmaceutically acceptable acid addition salt thereof, which comprises including one or more parabens in such solution, and adjusting the pH to range from about 5 to about 7.

12. Capsules, including but not limited to soft capsules or hard capsules, particularly soft gelatin capsules and hard gelatin capsules, comprising the oral solution according to claim 1.
